(19) United States
(12) Patent Application Publication
(10) Pub. No.: US 2004/0015198 A1
(43) Pub. Date: Jan. 22, 2004

(54) Method and apparatus for reducing heat flow

(75) Inventors: Paul M. Skarstad, Plymouth, MN (US); Kurt J. Casby, Grant, MN (US)

Correspondence Address: MEDTRONIC, INC., 710 MEDTRONIC PARKWAY NE, MS-LC340, MINNEAPOLIS, MN, 55432-5604, US

(73) Assignee: Medtronic, Inc.

(21) Appl. No.: 10/198,016

(22) Filed: Jul. 18, 2002

(51) Int. Cl. A61N 1/375

(52) U.S. Cl. 607/36

(57) ABSTRACT

The present invention provides a method and apparatus for reducing heat flow in sealed devices. The apparatus includes a casing adapted to enclose a volume. The apparatus further includes at least one sacrificial member positioned in the casing, wherein the at least one sacrificial member is adapted to absorb at least a portion of a heat flow in the volume.

### BACKGROUND OF THE INVENTION

1. Field of the Invention

This invention relates generally to sealed devices, and, more particularly, to reducing heat flow during the sealing process.

2. Description of the Related Art

Many devices may be hermetically sealed in a case to limit the interaction of the devices with environmental elements outside of the case. In some instances, it may be desirable to seal the case to protect electronic components in the device from damage. One example is an underwater flash camera. If water penetrates the camera case, the water could electrically short circuit capacitors that store energy for eventual discharge into the camera's flash bulb. In other instances, the hermetic seal may be used to protect elements in the environment surrounding the device from damage. For example, electronic devices that may be implanted in the human body, such as heart pacemakers, are generally hermetically sealed to reduce the chance that a patient might receive an unexpected shock if the patient's bodily fluids penetrate the device.

Heart pacemakers were first implanted in a human body in the 1960s. Thanks to the rapid pace of innovation in both the electronic and medical fields since then, doctors now have access to a wide assortment of body-implantable electronic medical devices including pacemakers, cardioverters, defibrillators, neural stimulators, and drug administering devices, among others. Millions of patients have benefited from, and many may owe their lives to, the proven therapeutic benefits of these devices.

Many body-implantable medical devices may receive operational power from an internal power source, such as a battery. The battery may serve a variety of functions, including, but not limited to, supplying power to electronic components of the device and charging capacitors that may discharge through electric leads into the heart to regulate heart rhythms. Smaller batteries generally lead to smaller devices, which may be less invasive and cause less patient discomfort. Therefore, much effort has been devoted to reducing the size of the batteries used in these devices. But the battery may contain electrically active or toxic materials, so it may be desirable to hermetically seal the battery. However, hermetically sealing the battery may increase the size of the battery.

Batteries in implantable devices are usually hermetically sealed by conforming a cover onto a casing that may contain one or more battery components using one or more of a plurality of methods, such as welding. However, many processes such as welding may generate a temperature gradient that may create a flow of heat between the cover and the one or more battery components in the casing. The heat flow may damage the one or more battery components. One such component is an insulator that may be used to reduce the chance of short-circuits by separating positively and negatively charged battery components. Although a refractory element, or “heat shield,” formed of ceramic, mica, or other heat-resistant polymers may be inserted between the source of the heat gradient and the battery components to deflect heat. However, a heat shield may only be partially effective. Therefore, it is generally prudent to add space between the point of conformity and the battery components to reduce the amount of heat that may reach the battery components.

To further protect the battery components from the heat that may be generated by the conforming process, such processes may be performed at a slower speed. However, this method may reduce the efficiency of the battery fabrication process. Lower production efficiency may raise the cost of the implantable medical devices, which may in turn limit the availability of these devices, further increasing patient discomfort.

The present invention is directed to overcoming, or at least reducing the effects of, one or more of the problems set forth above.

### SUMMARY OF THE INVENTION

In one aspect of the instant invention, an apparatus is provided for reducing heat flow in sealed devices. The apparatus includes a casing adapted to enclose a volume. The apparatus further includes at least one sacrificial member positioned in the casing, wherein the at least one sacrificial member is adapted to absorb at least a portion of a heat flow in the volume.

In one aspect of the present invention, a method is provided for reducing heat flow in sealed devices. The method includes applying heat to substantially seal a casing. The method further includes positioning a sacrificial member in the casing, wherein the sacrificial member is adapted to absorb at least a portion of said heat.

### BRIEF DESCRIPTION OF THE DRAWINGS

The invention may be understood by reference to the following description taken in conjunction with the accompanying drawings, in which like reference numerals identify like elements, and in which:

FIG. 1 schematically illustrates one embodiment of a system, in accordance with one embodiment of the present invention;

FIG. 2 illustrates a three-dimensional depiction of an implantable medical device that may be employed in the system of FIG. 1, in accordance with one embodiment of the present invention;

FIG. 3 shows a cross-sectional view of a battery that may be used to power components of the implantable medical device illustrated in FIG. 2, in accordance with one embodiment of the present invention; and

FIG. 4 shows an alternative embodiment of an apparatus for controlling heat flow in the battery of FIG. 3, in accordance with one embodiment of the present invention.

### DETAILED DESCRIPTION OF SPECIFIC EMBODIMENTS

Illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

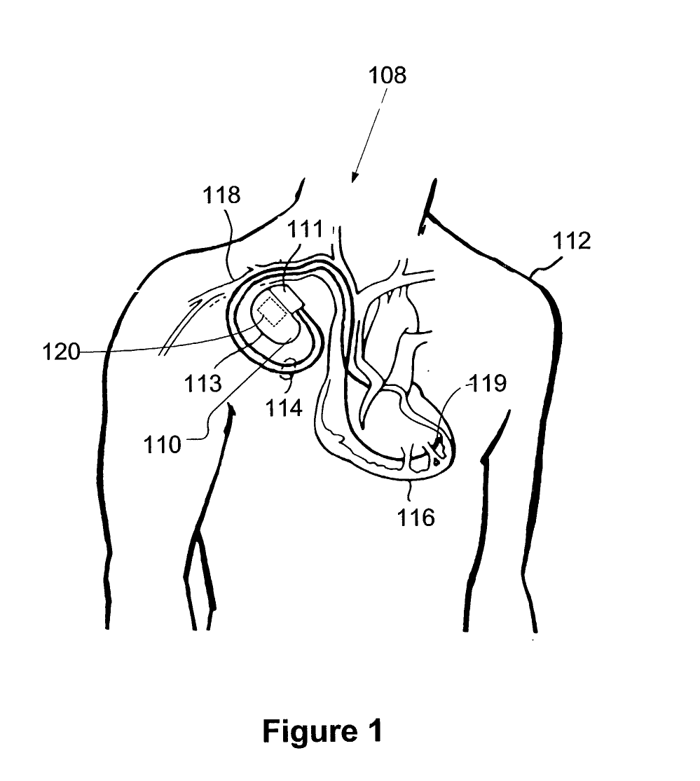

Referring now to FIG. 1, a stylized diagram of a system 108 in accordance with one embodiment of the present invention is presented. The system 108 comprises an implantable medical device 110, such as an implantable cardioverter defibrillator, that has been surgically implanted in a patient 112. The implantable medical device 110 may be housed within a hermetically-sealed, biologically inert casing 113. The term “hermetically-sealed,” as utilized herein, should be understood to mean tightly sealed against air and liquids, in accordance with standard usage as defined by Webster's Dictionary.

One or more leads, collectively identified with reference numeral 114 in FIG. 1 are electrically coupled to the implantable medical device 110 in a conventional manner and extend into the patient's heart 116 through a vein 118. Disposed generally near an end 119 of the leads 114 are one or more exposed conductive electrodes (not shown) for receiving electrical cardiac signals or delivering electrical pacing stimuli to the heart 116. In one embodiment, the implantable medical device 110 may administer a therapy that may reduce fibrillations in the heart 116. The implantable medical device 110 may also collect and store physiological data from the patient 112. The physiological data may include oxygen concentration in the blood, blood pressure, and cardiac electrogram signals.

Electrical power for the operation of the implantable medical device 110, including delivering therapy and acquiring physiological data, is generally provided by an internal battery 120. If the battery 120 should fail or malfunction, the function of the implantable medical device 110 may be compromised and it may be necessary to perform additional surgical procedures to remove the battery 120 from the patient 112. Therefore, it may be desirable to reduce the potential for damage to the battery 120 prior to placing the battery 120 in the patient 112, by implementing one or more embodiments of the present invention.

Since the battery 120 may be adapted to function in the patient 112, the battery 120 may be subject to other constraints. The internal battery 120 may, for example, contain electrically active or toxic components that may be harmful to the patient 112 and so it may be desirable to hermetically seal the battery to reduce the chance that the electrically active or toxic components leak out of the battery 120. It may also be desirable to hermetically seal the battery 120 to reduce the chance that bodily fluids penetrate into the battery 120, where they may damage the battery 120 and compromise the function of the implantable medical device 110. The implantable medical device 110 may also be uncomfortable for the patient 112. To reduce the discomfort of the patient 112, it may be desirable to reduce the size of the implantable medical device 110 by, for example, making the battery 120 smaller.

In view of the importance of the battery 120 to the function of the implantable medical device 110 and the potential benefits to the patient 112 of the implantable medical device 110, it may be desirable to improve methods of making the battery 120 to reduce the chance that the battery 120 may be damaged. It may also be desirable to reduce the size of the battery 120 and to hermetically seal the battery 120. In accordance with one embodiment of the present invention, and as explained in more detail below, the battery 120 may be hermetically sealed in such a way that damage to the battery 120 may be reduced and the size of the battery 120 may also be reduced.

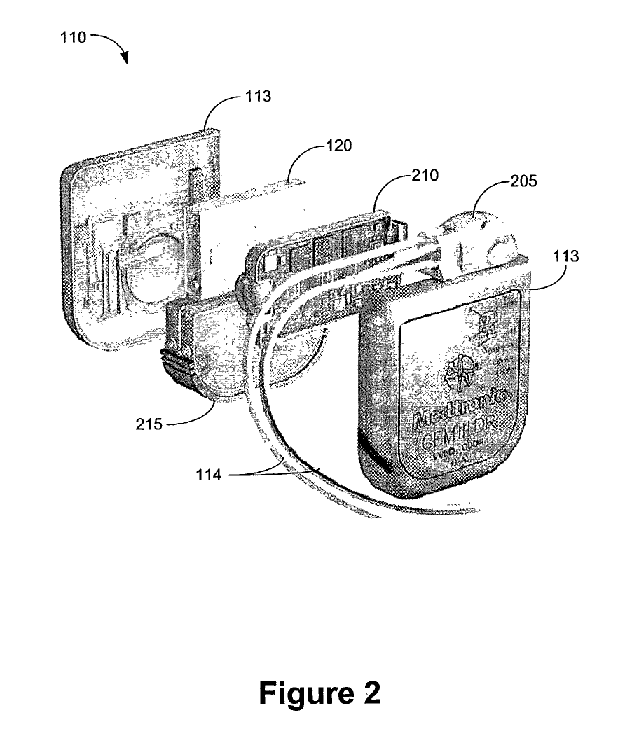

Turning now to FIG. 2, a stylized three-dimensional depiction of the implantable medical device 110 in accordance with one embodiment of the present invention is illustrated. In one embodiment, a casing 113 may include a variety of elements including, but not limited to, a connector 205, a processor unit 210, a capacitor package 215, and a battery 120. The elements in the casing 113 may be positioned in any of a variety of locations. The capacitor package 215 and the battery 120 may be electrically coupled to the processor unit 210. The leads 114 may be interfaced with the implantable medical device 110 through the connector 205 and may electrically connect portions of the patient 112 such as the heart 116 to the implantable medical device 110.

The processor unit 210 may detect and/or record electric cardiac signals that may travel from the heart 116 along the leads 114 and enter the implantable medical device 110 through the connector 205. The processor unit 210 may use the electric cardiac signals to determine when a cardiac event, such as a slow or erratic heart rate, occurs. In response to such a cardiac event or other conditions, the processor unit 210 may administer electric pacing stimuli to the heart 116 by releasing energy stored in the capacitor package 215 and directing the energy through the connector 205 and travel along the leads 114 to the heart 116. The capacitor package 215 may comprise one or more capacitors (not shown) that may store sufficient charge, such that when the charge is released, it can provide a cardiac therapy.

The battery 120 provides energy that may be used to power the processor unit 210 and to recharge the capacitor package 215 between electric pacing stimuli. Although it may be desirable to hermetically seal the battery 120, heat from the sealing process may damage the battery 120. Thus, in accordance with one embodiment of the present invention, and as explained in more detail below, heat flow in the battery 120 may be, at least partially, controlled during the sealing process. It should, however, be appreciated that the present invention may be advantageously embodied in numerous other systems in which it is desirable to form a hermetically-sealed environment. Such systems may include other implantable medical devices like heart pacemakers and drug delivery devices, as well as non-medical hermetically-sealed devices that may contain heat-sensitive components, such as capacitors and underwater devices.

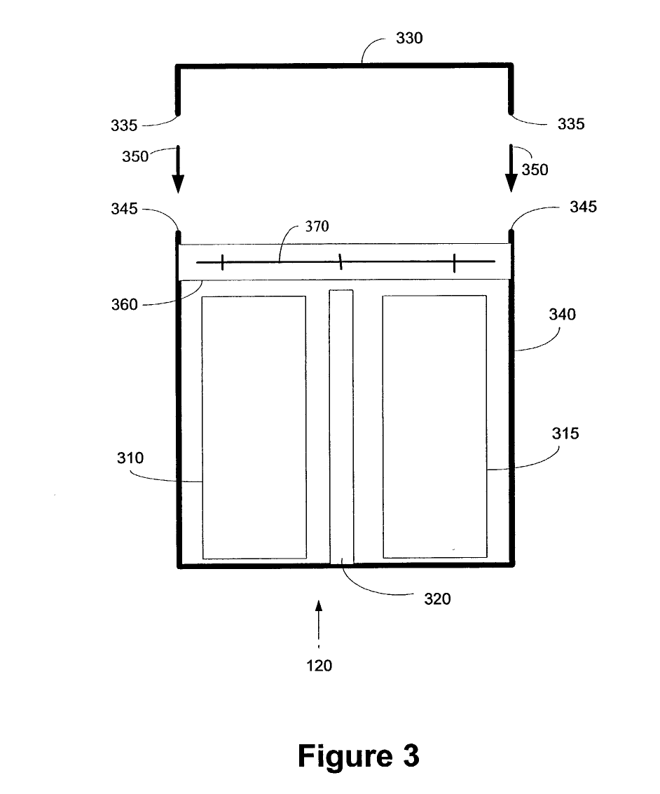

Turning now to FIG. 3, a stylized cross-sectional view of the battery 120 is depicted. The battery 120 may be comprised of an anode 310, a cathode 315, and a separator 320 that may be used to electrically isolate the anode 310 from the cathode 315. The battery 120 may contain additional elements (not shown) that facilitate electrical and electro-chemical reactions. In one embodiment, the separator may be formed of Celgard™ 2500, a microporous polypropylene sheet material, and the anode 310 and the cathode 315 may be formed of electrically active materials, such as lithium, combination silver vanadium oxide, carbon monofluoride (CFx), and the like. The active materials may cause damage to the patient 112 if they come in contact with elements of the human body. The anode 310 and the cathode 315 may also be damaged by contact with elements of the human body, such as bodily fluids. Thus, it may be desirable to hermetically seal the battery 120 to prevent, or at least significantly reduce, contact between the anode 310, the cathode 315, and elements of the body in which the battery 120 is implanted.

Therefore, in one embodiment, the anode 310, the cathode 315, the separator 320, and other battery elements (not shown) may be enclosed by a battery cover 330 bordered by an edge 335 and a battery casing 340 that may be similarly bordered by an edge 345. The battery cover 330 and/or the battery casing 340 may be formed from titanium, stainless steel, or other suitable materials. To hermetically seal the battery 120, the edge 335 of the battery cover 330 may be brought into contact with the edge 345 of the battery casing 340 by moving the battery cover 330 along the direction indicated by the arrows 350 in FIG. 3. The edges 335 and 345 may then be hermetically sealed. For example, a laser may be used to weld the battery cover 330 to the battery casing 340 by heating the edges 335 and 345 to temperatures near or above their respective melting points to hermetically seal the edges 335 and 345. Titanium, for example, may be used in one embodiment to form the casing and cover. Titanium has a melting point of about 1670° Centigrade (° C.). It should be noted, however, that in other embodiments, the hermetic seal between the edges 335 and 345 might be created using other techniques known to those skilled in the art having benefit of the present disclosure.

Due to the heat that may be generated during the sealing process, a temperature gradient may form in the battery 120. The edge 335 of the battery cover 330 and the edge 345 of the battery casing 340 may reach temperatures that are much higher than the typical temperatures of the remainder of the battery 120 resulting in the temperature gradient. For example, in one embodiment, the battery cover 330 may be formed of titanium, and consequently may reach temperatures near 1670° C., the approximate melting point of titanium. The temperature of the separator 320, on the other hand, may be lower. For example, in one embodiment, the separator 320 may be formed of Celgard™ 2500, a micro-porous polypropylene sheet material that has an onset of melting at approximately 115° C. and a peak melting temperature of approximately 175-180° C.

The temperature gradient may have a component directed from the point of the seal to elements within the battery casing 340 that may drive a flow of heat in the battery 120 during the sealing process. The heat flow may raise the temperature of portions of the battery 120 to a level high enough to damage one or more of the anode 310, the cathode 315, the separator 320, or other elements in the battery 120. If, for example, the temperature inside the battery casing 340 should approach or exceed the melting point of the separator 320, the separator 320 may melt. If the shape of the separator 320 changes due to melting, the separator may no longer keep the anode 310 and cathode 315 electrically isolated and may allow the anode 310 and cathode 315 to come into electrical contact, perhaps short-circuiting the battery 120. A rise of the temperature in the battery casing 340 may also damage the anode 310 and/or the cathode 315. Thus, it may be desirable to inhibit the flow of heat in the battery 120.

To reduce the heat flow from the edges 335 and 345 into the battery casing 340 during the sealing process, a sacrificial member 360 may be placed in the battery casing 340. For example, in one embodiment, the sacrificial member 360 may be positioned between the edge 345 and the active components of the battery 120, including, but not limited to, the anode 310, the cathode 315, and the separator 320. In alternative embodiments, however, the sacrificial member 360 may be positioned at one of numerous other desirable locations.

The sacrificial member 360 may at least partially melt and absorb heat flowing from the edges 335 and 345. Thus, by forming the sacrificial member 360 out of materials with an appropriate melting point, the sacrificial member 360 may disrupt the component of the temperature gradient directed from the point of the seal to the battery casing 340. Consequently, the increase in the temperature of the battery 120 during the sealing process may be substantially reduced, decreasing the chance that elements in the battery casing 340 may be damaged during the sealing process. For example, in one embodiment, the sacrificial member 360 may be formed of a crystalline material such as polyethylene. Heat is dissipated by melting crystallites (not shown) in the crystalline material. In the case of polyethylene, which has a melting point of about 120° C., the temperature of the polyethylene will remain at 120° C. for the duration of the melting process.

Furthermore, a conductive member 370 may be included in the sacrificial member 360. The conductive member 370 may, in alternative embodiments, be formed of carbon fiber, metallic struts, or other like materials. The conductive member 370 may collect heat and direct the heat to other areas of the sacrificial member 360, thus increasing the total volume of the material able to dissipate heat by melting crystallites. For example, if the average polymer crystallinity is 65% and the enthalpy of melting crystallites is 300 J/g in the sacrificial member 360, and radiant heat only melts a 5% percent of the total area of the sacrificial member 360, then if the sacrificial member 360 has a mass of one gram, approximately 10J will be dissipated. By directing the heat throughout the sacrificial member 360 with the conductive member 370, more crystallites can be melted and the total heat dissipated may increase. For example, if heat can be directed with the conductive member 370 within the polymeric sacrificial member 360 to melt 50% of the member, then, in the above example, the total heat dissipated may be increased by a factor of 10.

The present invention is not, however, limited to the sacrificial member 360 being formed of a crystalline material such as polyethylene. In alternative embodiments, the sacrificial member 360 may be formed of polymeric material, which may be formed by a variety of processes well known to those of ordinary skill in the art including, but not limited to injection, compression, solvents, and the like. The polymeric material may also include branched content (not shown) formed of olefin. The sacrificial member 360 may also be formed of a crystalline impregnated fabric.

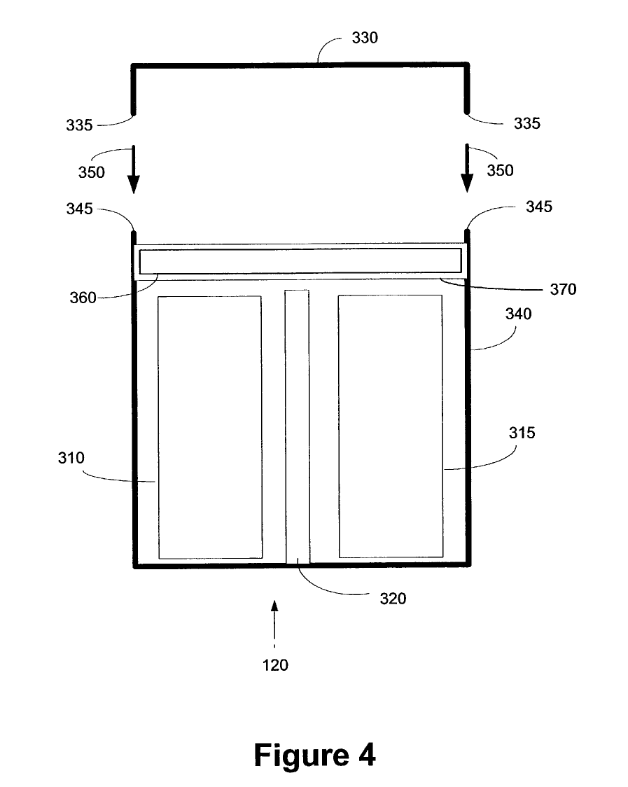

Referring now to FIG. 4, an alternate embodiment of an apparatus for controlling heat flow in the battery 120 is illustrated. In some casings, the sacrificial member 360 may deform during melting. Deformations in the shape of the sacrificial member 360 may reduce its heat absorption efficiency by, for example, the formation of openings in the sacrificial member 360. Portions of the sacrificial member 360 may also dislodge and penetrate the battery casing 340 during melting. Dislodged portions of the sacrificial member 360 may damage components in the battery casing 340 (e.g., the anode 310, the cathode 315, or the separator 320) by heating or otherwise reacting with the components. It may thus be desirable to support the sacrificial member 360 with a supporting member 370.

It may also be desirable to form the supporting member 370 of a refractory material. Suitable materials may include ethylenetetrafluoroethylene, polypropylene, ceramics, mica, or a metallized layer such as metal foil. However, it should be appreciated that the supporting member 370 may be formed of any appropriate material known to those skilled in the art having benefit of the present disclosure. Positioning the sacrificial member 360 in a supporting member 370 may improve the effectiveness of the sacrificial member 360 by reflecting some of the radiant heat flowing from the edges 335, 345 and further disrupting a component of the temperature gradient directed from the point of seal to the battery casing 340. Consequently, the increase in the temperature of the battery 120 during the sealing process may be substantially reduced by positioning the sacrificial member 360 in a supporting member 370 formed of a suitable refractory material, decreasing the chance that elements in the battery casing 340 may be damaged during the sealing process.

In some cases, portions of the sacrificial member 360 may liquefy and move through the battery 120, potentially damaging the anode 310, the cathode 315, or the separator 320. It may thus be desirable to enclose the sacrificial member 360 within the supporting member 370, although it should be appreciated that, in other embodiments, the supporting member 370 may have any desirable shape and may or may not fully enclose the sacrificial member 360. In alternative embodiments, it may also be desirable to position additional supporting members 370 and sacrificial members 360 in the battery case 340 in such a way that the temperature gradient may be further disrupted.

Thus, in accordance with one embodiment of the present invention, heat flow in the battery 120 may be, at least partially, controlled during the sealing process. It should, however, be appreciated that the present invention may be advantageously embodied in numerous other systems in which it is desirable to form a hermetically-sealed environment. Such systems may include other implantable medical devices like heart pacemakers, implantable pulse generators, and drug delivery devices, as well as non-medical hermetically-sealed devices that may contain heat-sensitive components, such as capacitors and underwater devices. The present invention may also be advantageously embodied in numerous other components of systems in which it is desirable to form a hermetically-sealed environment. Although not so limited, these components may include high-rate lithium batteries in deep drawn titanium casements, high-rate lithium batteries in shallow drawn titanium casements, medium-rate lithium batteries in deep drawn titanium casements, aluminum electrolytic capacitors in shallow drawn aluminum casements, and aluminum electrolytic capacitors in shallow drawn titanium casements

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. An apparatus, comprising:
a casing adapted to enclose a volume; and
at least one sacrificial member positioned in the casing, wherein the at least one sacrificial member is adapted to absorb at least a portion of a heat flow in the volume.

2. The apparatus of claim 1, wherein the at least one sacrificial member is adapted to absorb at least a portion of the heat flow by at least partially melting at least a portion of the sacrificial member.

3. The apparatus of claim 2, wherein the casing includes a cover adapted to form a seal with a case that substantially encloses the volume.

4. The apparatus of claim 3, wherein the casing is adapted to be hermetically sealed by the application of heat.

5. The apparatus of claim 4, wherein the sacrificial member is positioned substantially between the seal and the volume.

6. The apparatus of claim 5, wherein the casing is adapted to be implanted in a human body.

7. The apparatus of claim 6, wherein the volume substantially encloses at least one electronic component.

8. The apparatus of claim 1, further comprising at least one supporting member adapted to support the at least one sacrificial member, wherein the supporting member is adapted to reflect heat.

9. The apparatus of claim 8, wherein the at least one supporting member encloses the at least one sacrificial member.

10. The apparatus of claim 1, wherein the sacrificial member is formed of at least one of a crystalline material, a polymer, and a crystalline impregnated fabric.

11. The apparatus of claim 1, wherein the sacrificial member comprises at least one conductive member.

12. The apparatus of claim 11, wherein the conductive member is formed of at least one of carbon fiber and metal struts.

13. An apparatus, comprising:
a casing;
a cover adapted to form a seal with the casing using heat; and
at least one sacrificial member positioned in the casing, wherein the sacrificial member is adapted to absorb at least a portion of a heat flow by at least partially melting at least a portion of the sacrificial member.

14. The apparatus of claim 13, wherein the sacrificial member is adapted to reduce the heat flow from the seal to the volume.

15. The apparatus of claim 14, wherein the seal includes a hermetic seal.

16. The apparatus of claim 15, wherein the casing is adapted to be implanted in a human.

17. The apparatus of claim 16, wherein the volume substantially encloses at least one electronic component.

18. The apparatus of claim 17, wherein the at least one electronic component is a battery.

19. The apparatus of claim 18, wherein the at least one electronic component is a capacitor.

20. The apparatus of claim 13, further comprising at least one supporting member adapted to support the at least one sacrificial member, wherein the at least one supporting member is adapted to reflect heat.

21. The apparatus of claim 20, wherein the at least one supporting member encloses the at least one sacrificial member.

22. A method, comprising:
applying heat to substantially seal a casing; and
positioning a sacrificial member in the casing, wherein the sacrificial member is adapted to absorb at least a portion of said heat.

23. The method of claim 22, wherein absorbing at least a portion of said heat comprises at least partially melting at least a portion of the sacrificial member.

24. The method of claim 23, wherein substantially sealing the casing comprises substantially sealing an edge of a cover in alignment with an edge of the casing.

25. The method of claim 24, wherein positioning the sacrificial member further comprises positioning the sacrificial member substantially between the cover and the casing.

26. The method of claim 22, further comprising supporting the at least one sacrificial member with at least one supporting member being adapted to reflect heat.

27. The method of claim 26, wherein supporting the sacrificial member further comprises enclosing the sacrificial member in the supporting member.

28. The method of claim 23, wherein sealing the casing further comprises hermetically sealing the casing.

29. The method of claim 23, further comprising forming the at least one sacrificial member with at least one conductive member being adapted to conduct heat through the sacrificial member.

30. A method, comprising:
providing a casing to enclose at least one electronic component; and
providing at least one of a heat absorber, a heat conductor, and a heat insulator, wherein the at least one of a heat absorber, a heat conductor, and a heat insulator is adapted to reduce the flow of heat in the casing by at least partially melting at least a portion of the heat absorber.

31. The method of claim 30, wherein providing the casing comprises sealing an edge of a cover to an edge of the casing substantially enclosing the electronic component.

32. The method of claim 31, wherein sealing comprises hermetically sealing the edge of the cover to the edge of the casing.

33. The method of claim 32, further comprising adapting the casing to be implanted in a living being.

34. An apparatus, comprising:
a sealed casing enclosing at least one electronic component; and
a partially melted sacrificial material positioned in the sealed casing, wherein the partially melted sacrificial member is capable of absorbing a portion of heat.

35. The apparatus of claim 34, wherein the electronic component is a battery.

36. The apparatus of claim 34, wherein the electronic component is a capacitor.

37. The apparatus of claim 34, wherein the sacrificial member further comprises a refractory supporting member.

38. The apparatus of claim 34, wherein the sacrificial member comprises branched content formed of olefin.
